(19) United States
(12) Patent Application Publication
(10) Pub. No.: US 2009/0196866 A1
(43) Pub. Date: Aug. 6, 2009

(54) Pharmaceutical composition containing an iron-binding agent

(76) Inventor: Arne Hengerer, Erlangen (DE)

Correspondence Address: HARNESS, DICKEY & PIERCE, P.L.C., P.O.BOX 8910, RESTON, VA, 20195, US

(21) Appl. No.: 12/230,770

(22) Filed: Sep. 4, 2008

(30) Foreign Application Priority Data
Sep. 5, 2007 (DE) 10 2007 042 107.0

(51) Int. Cl. A61K 38/46; A61K 38/16; C07H 21/00; A61P 35/00

(52) U.S. Cl. 424/94.67; 514/12; 514/6; 536/23.1

(57) ABSTRACT

A pharmaceutical composition is disclosed. In at least one embodiment, a pharmaceutical drug is linked via a peptide linker to an iron-binding agent, for example an iron-binding protein, with the iron-binding agent in turn being bound to an iron-containing magnetic particle. The peptide linker has a protease recognition sequence, i.e. it may be cleaved at said protease recognition sequence by a protease, thereby enabling the pharmaceutical drug to be released locally. The pharmaceutical composition is suitable for magnetic drug targeting.

### PRIORITY STATEMENT

The present application hereby claims priority under 35 U.S.C. §119 on German patent application number DE 10 2007 042 107.0 filed Sep. 5, 2007, the entire contents of which is hereby incorporated herein by reference.

### FIELD

Embodiments of the invention generally relate to a pharmaceutical composition having an iron-binding agent and a pharmaceutical drug. Embodiments of the invention further generally relate to a corresponding nucleic acid.

### BACKGROUND

A key problem in the chemotherapeutic treatment of tumors or other pathologies is the fact that the pharmaceutical drugs used (for example cytostatic agents) have severe side effects. Such is the prominence of this problem that tumor patients in particular often die from the severe side effects of chemotherapy rather than from the disease.

One approach to solving this problem resides in concentrating the pharmaceutical drugs locally at the site of action, for example at the tumor, thereby achieving a higher dosage locally while reducing the side effects at the same time. One strategy used in this context is magnetic drug targeting: this involves coupling pharmaceutical drugs to iron oxide nanoparticles which are also known as contrast media for nuclear spin tomography imaging. Thus, for example, Alexiou et al. let the chemotherapeutic drug mitoxantrone adsorb to coated superparamagnetic iron oxide nanoparticles and injected this composition in suspension as a medicament (“Loco regional Cancer Treatment with magnetic drug Targeting”, 2000, Cancer Research, 60, 6641-6648).

The injected composition can be “focused” at the site of action, for example at a tumor, by an external magnetic field. At the site of action, mitoxantrone desorbs from the coated iron oxide nanoparticles and can act locally on the tumor.

Coupling the medicament to the inorganic iron oxide nanoparticle is not trivial and is a critical step in the synthesis of the pharmaceutical composition. Previously the pharmaceutical drug has been coupled to the iron oxide nanoparticle via a coating layer of said nanoparticle. Biopolymers (PEG, polysaccharides, for example starch and the like) are used in the coating layer. Since the active compound is bound to the nanoparticle only in weak association and has a poorly defined 3D structure, the coupling is not very stable. In contrast, if the active compound is coupled covalently to the iron oxide nanoparticles, binding is too strong and release of the active compound cannot be guaranteed.

### SUMMARY

In at least one embodiment of the present invention, a pharmaceutical composition is provided which enables a pharmaceutical drug to be stably coupled to magnetic particles but which at the same time enables or ensures release of the active compound in a reliable manner at the site of action.

In at least one embodiment, the active compound, rather than being coupled to iron-containing, magnetic particles in an unspecific, associative manner and/or by absorption, is linked via a peptide linker to an iron-binding agent, for example an iron-binding protein, with the iron-binding agent in turn being bound to the iron-containing magnetic particle. The peptide linker has a protease recognition sequence, i.e. it can be cleaved by a protease at this protease recognition sequence, thereby enabling the pharmaceutical drug to be released locally.

In at least one embodiment, the invention utilizes the circumstance that the cellular surface has proteases which cleave particular recognition sequences. This is advantageous in particular because many tumor cells overexpress certain proteases, for example matrix metalloproteinases (MMPs). Thus it is possible, for example, to choose a protease recognition sequence which is recognized and cut by a particular protease which is overexpressed by a tumor, for example.

In this way, the pharmaceutical composition of at least one embodiment of the invention can be tailored to the particular application. This makes possible an additional release which is specifically increased at the site of action.

The term peptide linker means a short amino acid sequence via which the active compound is coupled to the iron-binding agent. This amino acid sequence of the peptide linker may be, for example, 10-20 amino acids in length, and a person skilled in the art is familiar with customary amino acid sequences which can be used as peptide linkers, for example polyglycine sequences and the like. The amino acid sequence of the peptide linker has, according to the invention, also a protease recognition sequence. Proteases, i.e. enzymes capable of cleaving proteins, often recognize particular sequence motifs. These sequence motifs for the particular proteases are known to the skilled worker or can simply be determined by literature searches or database queries.

Tumors are in particular known to express frequently matrix metalloproteinases (MMPs) whose activity plays an important part in the spreading of the tumor. See, for example, Visse et al., “Matrix Metalloproteinases and Tissue Inhibitors of Metalloproteinases”, Circulation Research, 2003, 92, 827-839), the entire contents of which is hereby incorporated herein by reference. The MMPs are either secreted by the cells or anchored within the cellular membrane. The matrix metalloproteinases constitute a family of related proteins which usually cleave a peptide bond in front of an amino acid residue having a hydrophobic side chain, for example leucine, isoleucine, methionine, phenylalanine or tyrosine.

A number of protease recognition sequences for matrix metalloproteinases have been defined, see, for example, Visse et al., Chen et al., “A unique substrate recognition profile for matrix metalloproteinase-2.”, Journal of Biological Chemistry, 2002, 277, 4485-4491 or Deng et al., “Substrate specificity of human collagenase 3 assessed using a phage-displayed peptide library”, Journal of Biological Chemistry, 2000, 275, 31422-31427,), the entire contents of each of which is hereby incorporated herein by reference. The protease recognition sequences and motifs specified in Visse et al., Deng et al. and Chen et al. are examples of suitable protease recognition sequences for the present invention. Targeting active compounds by means of magnetic drug targeting, which involves a proteinase cleaving and releasing the active compound, has the advantage that the active compound can in this way stably bind to the magnetic particle but, at the same time, is guaranteed to be released, particularly advantageously, if the site of action is a tumor whose cells strongly express the corresponding proteinase or proteinases, thereby rendering said targeting more specific.

It is noted that the term “iron-binding” is not limited to the binding of elemental iron but comprises binding of the element iron in any form, as element, as ion, as oxide, in the form of particles or nanoparticles, etc.

Preference is given to the iron-binding agent having a first polypeptide, said polypeptide having a first amino acid sequence of a bacterial iron-binding protein or a derivative of said first amino acid sequence, which has an iron-binding activity.

A polypeptide has a defined sequence of amino acids. An isolated polypeptide means, in the context of the present invention, a polypeptide which is present in a form which is essentially free of other polypeptides.

A bacterial iron-binding protein means a protein which binds iron with high specificity (for example a binding constant of >10¹⁵ mol⁻¹, preferably >10¹⁸ mol⁻¹) and which is of bacterial origin. A derivative of a bacterial iron-binding protein means a polypeptide derived from the natural protein, which has sequence homology to the natural protein and which itself has an iron-binding activity with a high binding constant (for example a binding constant of >10¹⁵ mol⁻¹, preferably >10¹⁸ mol⁻¹).

Iron is an important trace element for living organisms, this being the result firstly of the abundance of this element in the environment and secondly of its chemical properties, since iron has two stable oxidation states (+II/+III) which are interconvertible and suitable for participation in redox processes, for example within the respiratory chain. Iron may be present in proteins in different structures, for example as heme group, as iron-sulfur cluster, as iron nickel, as diiron or as mononuclear iron. As cofactor of many enzymes it is involved in important metabolic processes. However, due to the low solubility products of Fe(II) and Fe(III), it is difficult for cells to obtain.

In addition, Fe(III) hydrolyzes in an aqueous environment and forms hydroxide polymers which may precipitate under physiological conditions. Since iron also catalyzes the formation of free radicals, it has moreover highly toxic effects. For this reason, the availability of iron in the body is strictly regulated, and iron is bound and may be transported into the cell by endogenous iron-binding proteins, for example transferring, ferritins.

Iron is a valuable resource for bacteria which colonize the body of a mammal. For this reason, bacteria have evolved a multiplicity of iron-binding proteins which in turn serve to bind iron and make it available for bacteria. These bacterial iron-binding proteins are distinguished by extremely high iron binding constants, for example >10¹⁵ mol⁻¹ or >10¹⁸ mol⁻¹ (Briat J.-F., 1992, “Iron assimilation and storage in prokaryotes”, Journal of General Microbiology, 138, 2475-2483, the entire contents of which is hereby incorporated herein by reference). Accordingly, bacterial iron-binding proteins are extremely efficient iron chelators.

The bacterial iron-binding protein is preferably a siderophore. Siderophores are high-affinity extracellular iron(III) chelators.

Preference is given to the bacterial iron-binding protein being an Fe(III)-binding protein (Fbp) or a major ferric iron binding protein (MIRP) of the Haemophilus, Pasteurellales, Pasteurellaceae or Neisseria family. More preference is given to the bacterial iron-binding protein being an Fe(III)-binding Protein (Fbp) of the species H. influenzae, N. gonorrhoeae, N. meningitidis, N. cinerea, N. lactamica, N. subflavia, N. kochii or N. polysaccharea.

According to an embodiment of the invention, the first amino acid sequence is chosen from the group consisting of:
a) the amino acid sequence according to SEQ ID NO:1 or SEQ ID NO:2;
b) an amino sequence which has at least 15, preferably 30, consecutive amino acids of the amino acid sequence according to SEQ ID NO:1 or SEQ ID NO:2;
c) an amino acid sequence of a derivative of a polypeptide having an amino acid sequence according to SEQ ID NO:1 or SEQ ID NO:2, said derivative being encoded by a nucleic acid molecule which hybridizes under stringent conditions to a nucleic acid molecule which encodes a polypeptide having the amino acid sequence according to SEQ ID NO:1 or SEQ ID NO:2;
d) an amino acid sequence which is at least 60% homologous to the amino acid sequence according to SEQ ID NO:1 or SEQ ID NO:2; and
e) an amino acid sequence of a derivative of a polypeptide having the amino acid sequence according to SEQ ID NO: 1 or SEQ ID NO: 2, said derivative being encoded by a nucleic acid molecule which is at least 60% homologous to a nucleic acid molecule which encodes the polypeptide having the amino acid sequence according to SEQ ID NO:1 or SEQ ID NO:2.

In the context of embodiments of the invention, stringent hybridization conditions mean conditions which enable hybridization of allelic variants but not hybridization with other non-related genes. The usual conditions known to a person skilled in the art are used herein, as described in Sambrook et al. (Molecular cloning. A Laboratory Manual, Cold Spring Harbor Laboratory Press, 2nd edition, 1989, the entire contents of which is hereby incorporated herein by reference). Examples of stringent hybridization conditions are six times sodium chloride/sodium citrate (6×SSC) at about 45° C., followed by a washing step with 2×SSC at 50° C. In the context of embodiments of the present invention, the term “homologous” means a defined homology of at least 60%, preferably 75%, more preferably 90%, at the DNA or amino acid level, which homology may be determined by known methods, for example by computer-assisted sequence comparisons. This involves comparing by computer two sequences to be studied with respect to their homology in such a way (e.g. Altschul et al. [1999], Basic local alignment search tool, Journal of Molecular Biology, 215 or by the “global alignment program” [GAP] of Genetics Computer Group [GCG], the entire contents of which is hereby incorporated herein by reference) that the best possible match (alignment) is achieved, with the number of matching nucleotides or amino acids then being expressed as a percentage of said nucleotides or amino acids in the sequence.

At least one embodiment of the invention also relates to a nucleic acid molecule comprising a nucleic acid sequence which encodes a polypeptide of at least one embodiment of the invention, which polypeptide comprises an iron-binding protein or derivative thereof having iron-binding activity and the peptide linker. Said nucleic acid sequence may be present in a suitable vector, for example an expression plasmid suitable for a particular expression system. An isolated nucleic acid molecule means a nucleic acid molecule which is present in a form that is essentially free of other nucleic acids.

Preference is also given to the pharmaceutical composition of at least one embodiment of the invention having a pharmaceutically suitable carrier. The latter may comprise a suitable buffer, for example physiological saline, and the pharmaceutical composition may also contain suitable suspension agents and other pharmaceutical excipients as known to the skilled worker.

### BRIEF DESCRIPTION OF THE DRAWINGS

### Example of an Embodiment of the Invention

The invention will be illustrated below on the basis of an example embodiment and the appended drawings in which:

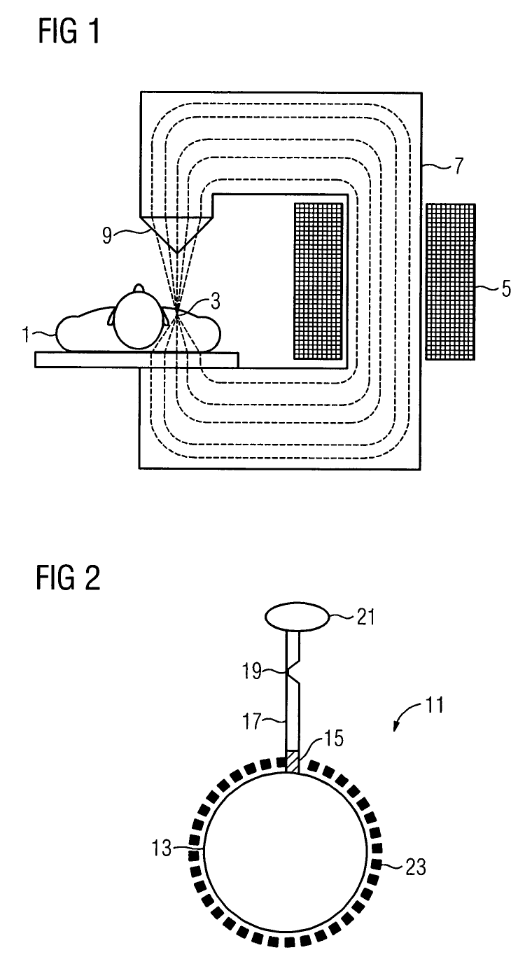

FIG. 1: is a diagrammatic representation of the principle of magnetic drug targeting;

FIG. 2: is a simplified, diagrammatic representation of a pharmaceutical composition of an embodiment of the invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

In magnetic drug targeting, as depicted in FIG. 1, a patient 1 is introduced into a magnetic field 3. The magnetic field 3 is generated by a coil 5 in a coil core 7 and can be focused with the aid of a specially adapted pole shoe 9. In this way, magnetic particles in the body of the patient are retained in the focus of the magnetic field 3. The patient is positioned in such a way that the focus of the magnetic field is in or at a body region to be treated, for example at a tumor growth.

Preparation of a Pharmaceutical Composition of an Embodiment of the Invention

To prepare a pharmaceutical composition of the invention, firstly a fusion polypeptide is provided which has the amino acid sequence according to SEQ ID NO: 1 bound to a peptide linker, for example a polyglycine sequence of approx. 10-20 amino acids, which is interrupted by a protease recognition sequence for a matrix metalloproteinase. A nucleic acid coding for the fusion peptide may be cloned in a suitable expression vector according to conventional cloning techniques known to the skilled worker (cf. Sambrook et al. Molecular cloning. A Laboratory Manual, Cold Spring Harbor Laboratory Press, 2nd edition, 1989, the entire contents of which are hereby incorporated herein by reference). The polypeptide may be expressed in a suitable expression system (e.g. E. coli, baculovirus, CHO cells or the like). The polypeptide is then purified by means of a suitable method, The polypeptide is subsequently coupled via the peptide linker to a chemotherapeutic agent, for example mitoxantrone (1,4-dehydroxy-5,8-bis[2-(2-hydroxyethylamino)ethylamino]-anthracene-9,10-dione), for example by a condensation reaction via one of the hydroxy groups of the active compound.

Preparation of the Iron Oxide Particles

To prepare the iron oxide particles, firstly a dispersion of monocrystalline or monodisperse iron oxide particles (nanoparticles of 1-100 nm in diameter) is prepared, in a 1-step synthesis, from iron(II) and iron(III) salts. An ammonia solution or an NaOH solution is added with stirring to an aqueous iron chloride solution (FeCl₂ and FeCl₃). Magnetite, Fe₃O₄, precipitates out of the solution. Oleic acid is added to the suspension with stirring and heating, resulting in a fine dispersion of magnetite particles (cf. also Park et al. (2005), One-Nanometer-Scale-Size-Controlled Synthesis of Monodisperse Magnetic Iron Oxide Nanoparticles, Angewandte Chemie International Edition, 44, 19, 2872-2877, the entire contents of which are hereby incorporated herein by reference).

The particles are purified and then, in a second step, coated with the fusion protein with the pharmaceutical drug coupled thereto, which fusion protein is titrated in an aqueous solution to the iron suspension with stirring. Finally, still unoccupied surfaces of the iron oxide nanoparticles must be saturated, for example with dextran, PEG, starch or the like. After purification, the coated particles are resuspended in an aqueous, pH-buffered solution. The iron concentration of the pharmaceutical composition should be, for example, from 0.1 mmol to 1.0 mmol Fe/ml.

The pharmaceutical composition prepared in this way may be administered to the patient by means of injection, for example via an infusion catheter. Since such a pharmaceutical composition acts as a contrast medium in a magnetic resonance imaging process, the concentration by magnetic drug targeting may also be monitored by way of MR tomography.

FIG. 2 depicts diagrammatically a molecule 11 of the pharmaceutical composition of the invention. An iron oxide nanoparticle 13 is linked via an iron-binding protein 15 and a peptide linker 17 with incorporated protease recognition sequence 19 to a pharmaceutical drug 21. Free surfaces of the iron particle 13 are saturated with a saturation agent 23 (e.g. PEG, starch). The drug 21 may be a chemotherapeutic agent, in particular an antibiotic, a cytostatic agent, a therapeutic antibody, an antimetabolite, a taxane, an alkaloid, a tyrosine kinase inhibitor, a biologically active peptide, and the like. The choice of therapeutic drug depends on the particular application and may be determined readily by a person skilled in the art, depending on the application. A person skilled in the art also knows that he can adapt the sequence of the peptide linker to the particular use purpose (choice of protease recognition sequence, choice and length of the peptide linker sequence, position of the protease recognition sequence within the peptide linker sequence, etc.).

It should be emphasized that the example embodiment is depicted merely by way of illustration and example. Many variations and modifications are conceivable within the scope of the invention, in particular with respect to the choice of iron-binding agent, peptide linker, protease recognition sequence and pharmaceutical drug. Further modifications with respect to the particular presentation of the pharmaceutical composition are likewise at the discretion of the skilled worker.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A pharmaceutical composition having
a) an iron-binding agent;
b) a pharmaceutical drug; and
c) a peptide linker via which said pharmaceutical drug is coupled to said iron-binding agent, wherein said peptide linker has a protease recognition sequence.

2. The pharmaceutical composition as claimed in claim 1, wherein the iron-binding agent has a first polypeptide, said polypeptide having a first amino acid sequence of a bacterial iron-binding protein or a derivative of said first amino acid sequence, which has an iron-binding activity.

3. The pharmaceutical composition as claimed in claim 2, wherein the bacterial iron-binding protein is a siderophore.

4. The pharmaceutical composition as claimed in claim 2, wherein the bacterial iron-binding protein is an Fe(III)-binding protein (Fbp) or a major ferric binding protein (MIRP) of the Haemophilus, Pasteurellales, Pasteurellaceae or Neisseria family.

5. The pharmaceutical composition as claimed in claim 2, wherein the bacterial iron-binding protein is an Fe(III)-binding Protein (Fbp) of the species H. influenzae, N. gonorrhoeae, N. meningitidis, N. cinerea, N. lactamica, N. subflava, N. kochii or N. polysaccharea.

6. The pharmaceutical composition as claimed in claim 2, wherein the first amino acid sequence is chosen from the group consisting of:
a) the amino acid sequence according to SEQ ID NO:1 or SEQ ID NO:2;
b) an amino sequence which has at least 15 consecutive amino acids of the amino acid sequence according to SEQ ID NO:1 or SEQ ID NO:2;
c) an amino acid sequence of a derivative of a polypeptide having the amino acid sequence according to SEQ ID NO:1 or SEQ ID NO:2, said derivative being encoded by a nucleic acid molecule which hybridizes under stringent conditions to a nucleic acid molecule which encodes the polypeptide having the amino acid sequence according to SEQ ID NO:1 or SEQ ID NO:2;
d) an amino acid sequence which is at least 60% homologous to the amino acid sequence according to SEQ ID NO:1 or SEQ ID NO:2; and
e) an amino acid sequence of a derivative of a polypeptide having the amino acid sequence according to SEQ ID NO: 1 or SEQ ID NO: 2, said derivative being encoded by a nucleic acid molecule which is at least 60% homologous to a nucleic acid molecule which encodes the polypeptide having the amino acid sequence according to SEQ ID NO:1 or SEQ ID NO:2.

7. The pharmaceutical composition as claimed in claim 1, wherein the peptide linker has a protease recognition sequence of a matrix metalloproteinase.

8. The pharmaceutical composition as claimed in claim 2, having a fusion polypeptide composed of peptide linker and first polypeptide.

9. The pharmaceutical composition as claimed in claim 1, wherein the pharmaceutical drug is a chemotherapeutic agent.

10. The pharmaceutical composition as claimed in claim 1, wherein the pharmaceutical drug is a cytostatic agent.

11. The pharmaceutical composition as claimed in claim 1, wherein the pharmaceutical drug is an antibody.

12. The pharmaceutical composition as claimed in claim 1, having iron oxide particles.

13. The pharmaceutical composition as claimed in claim 1, having a pharmaceutically suitable carrier.

14. A nucleic acid molecule which encodes a fusion polypeptide as claimed in claim 8.

15. The use of the fusion polypeptide as claimed in claim 8 for preparing a medicament.

16. The pharmaceutical composition as claimed in claim 3, wherein the bacterial iron-binding protein is an Fe(III)-binding protein (Fbp) or a major ferric binding protein (MIRP) of the Haemophilus, Pasteurellales, Pasteurellaceae or Neisseria family.

17. The pharmaceutical composition as claimed in claim 3, wherein the bacterial iron-binding protein is an Fe(III)-binding Protein (Fbp) of the species H. influenzae, N. gonorrhoeae, N. meningitidis, N. cinerea, N. lactamica, N. subflava, N. kochii or N. polysaccharea.

18. The pharmaceutical composition as claimed in claim 3, wherein the first amino acid sequence is chosen from the group consisting of:
a) the amino acid sequence according to SEQ ID NO:1 or SEQ ID NO:2;
b) an amino sequence which has at least 15 consecutive amino acids of the amino acid sequence according to SEQ ID NO:1 or SEQ ID NO:2;
c) an amino acid sequence of a derivative of a polypeptide having the amino acid sequence according to SEQ ID NO:1 or SEQ ID NO:2, said derivative being encoded by a nucleic acid molecule which hybridizes under stringent conditions to a nucleic acid molecule which encodes the polypeptide having the amino acid sequence according to SEQ ID NO:1 or SEQ ID NO:2;
d) an amino acid sequence which is at least 60% homologous to the amino acid sequence according to SEQ ID NO:1 or SEQ ID NO:2; and
e) an amino acid sequence of a derivative of a polypeptide having the amino acid sequence according to SEQ ID NO: 1 or SEQ ID NO: 2, said derivative being encoded by a nucleic acid molecule which is at least 60% homologous to a nucleic acid molecule which encodes the polypeptide having the amino acid sequence according to SEQ ID NO:1 or SEQ ID NO:2.

19. The pharmaceutical composition as claimed in claim 2, wherein the peptide linker has a protease recognition sequence of a matrix metalloproteinase.

20. The pharmaceutical composition as claimed in claim 2, wherein the pharmaceutical drug is a chemotherapeutic agent.

21. The pharmaceutical composition as claimed in claim 2, wherein the pharmaceutical drug is a cytostatic agent.

22. The pharmaceutical composition as claimed in claim 2, wherein the pharmaceutical drug is an antibody.

23. The pharmaceutical composition as claimed in claim 2, having iron oxide particles.

24. The pharmaceutical composition as claimed in claim 2, having a pharmaceutically suitable carrier.

25. The use of the nucleic acid as claimed in claim 14 for preparing a medicament.


SEQUENCE LISTING (2 sequences)

SEQ ID NO: 1 - protein, 332 aa, Haemophilus Influenzae
  ref <301>: AUTHORS: Harrison,A., Dyer,D.W., Gillaspy,A., Ray,W.C., Mungur,R.,
  ref <302>: TITLE: Genomic Sequence of an Otitis Media Isolate of Nontypeable
  ref <303>: JOURNAL: J. Bacteriol.
  ref <304>: VOLUME: 187
  ref <305>: ISSUE: 13
  ref <306>: PAGES: 4627-36
  ref <307>: DATE: 2005-07-01
  ref <308>: DATABASE ACCESSION NUMBER: YP_247820
  ref <309>: DATABASE ENTRY DATE: 2006-01-18
  ref <313>: RELEVANT RESIDUES IN SEQ ID NO: (1)..(332)
  ref <308>: DATABASE ACCESSION NUMBER: YP_247820
  ref <309>: DATABASE ENTRY DATE: 2005-06-27
  ref <313>: RELEVANT RESIDUES IN SEQ ID NO: (1)..(332)
  ref <308>: DATABASE ACCESSION NUMBER: NCBI/YP_247820
  ref <309>: DATABASE ENTRY DATE: 2005-06-27
  ref <313>: RELEVANT RESIDUES IN SEQ ID NO: (1)..(332)
  ref <308>: DATABASE ACCESSION NUMBER: NCBI / YP_247820
  ref <309>: DATABASE ENTRY DATE: 2005-06-27
  ref <313>: RELEVANT RESIDUES IN SEQ ID NO: (1)..(332)
MQFKHFKLATLAAALAFSANSFADITVYNGQHKEAATAVAKAFEQETGIKVTLNSGKSEQLAGQLKEEGDKTPADVFYTEQTATFADLSEAGLLAPISEQTIQQTAQKGVPLAPKKDWIALSGRSRVVVYDHTKLSEKDMEKSVLDYATPKWKGKIGYVSTSGAFLEQVVALSKMKGDKVALNWLKGLKENGKLYAKNSVALQAVENGEVPAALINNYYWYNLAKEKGVENLKSRLYFVRHQDPGALVSYSGAAVLKASKNQAEAQKFVDFLASKKGQEALVAARAEYPLRADVVSPFNLEPYEKLEAPVVSATTAQDKEHAIKLIEEAGLK

SEQ ID NO: 2 - protein, 330 aa, Neisseria meningitidis
  ref <301>: AUTHORS: Berish,S.A., Kapczynski,D.R. and Morse,S.A
  ref <302>: TITLE: Nucleotide sequence of the Fbp gene from Neisseria meningitidis
  ref <303>: JOURNAL: Nucleic Acids Res.
  ref <304>: VOLUME: 18
  ref <305>: ISSUE: 15
  ref <306>: PAGES: 4596
  ref <307>: DATE: 1990-06-01
  ref <308>: DATABASE ACCESSION NUMBER: NCBI / CAA 37561
  ref <309>: DATABASE ENTRY DATE: 1990-06-01
  ref <313>: RELEVANT RESIDUES IN SEQ ID NO: (1)..(330)
  ref <308>: DATABASE ACCESSION NUMBER: CAA37561
  ref <309>: DATABASE ENTRY DATE: 1990-06-01
  ref <313>: RELEVANT RESIDUES IN SEQ ID NO: (1)..(330)
  ref <308>: DATABASE ACCESSION NUMBER: NCBI / CAA37561
  ref <309>: DATABASE ENTRY DATE: 1990-06-01
  ref <313>: RELEVANT RESIDUES IN SEQ ID NO: (1)..(330)
MKTSIRYALLAAALTAATPALADITVYNGQHKEAAQAVADAFTRATGIKVKLNSAKGDQLAGQIKEEGSRSPADVFYSEQIPALATLSAANLLEPLPASTINETRGKGVPVAAKKDWVALSGRSRVVVYDTRKLSEKDLEKSVLNYATPKWKNRIGYAPTSGAFLEQVVAIVKLKGEAAALKWLKALKEYGKPYAKNSVALQAVENGEIDAALINNYYWHAFAREKGVQNVHTRLNFVRHRDPGALVTYSGAVLKSSQNKDEAKKFVAFLAGKEGQRALTAVRAEYPLNPHVVSTFNLEPIAKLEAPQVSATTVSEKEHATRLLEQAGMK